(19) United States
(12) Patent Application Publication
(10) Pub. No.: US 2007/0219219 A1
(43) Pub. Date: Sep. 20, 2007

(54) Methods of Preparation and Resolution of E/Z Isomers of Vinylfuro[2,3-D]Pyrimidine and their Biological Activities and related compositions and methods of treatment

(76) Inventors: Aleem Gangjee, (US); George R. Martin, (US); Kumar Gadamasetti, (US)

Correspondence Address: ECKERT SEAMANS CHERIN & MELLOTT, 600 GRANT STREET, 44TH FLOOR, PITTSBURGH, PA, 15219, US

(21) Appl. No.: 11/687,888

(22) Filed: Mar. 19, 2007

Related U.S. Application Data
(60) Provisional application No. 60/784,128, filed on Mar. 20, 2006.

(51) Int. Cl. A61K 31/519; C07D 491/02

(52) U.S. Cl. 514/260.1; 544/278

(57) ABSTRACT

Stereoselective methods for preparing the individual isomers, E- and Z-2,4-substituted-5-vinylfuro[2,3-d]pyrimidine and pharmaceutically acceptable salts, solvates, and prodrugs thereof using selective synthetic conditions are presented. This class of pyrimidine compounds function as receptor tyrosine kinase inhibitors during angiogenesis and resists the development of new blood vessels in tumors. These compounds also inhibit the folate pathway required for cell growth. The isomers of these compounds are resolved by physical, chromatographic, and/or HPLC methods. Their biological activities are described.

### CROSS REFERENCE TO RELATED APPLICATION

This application claims priority to U.S. Provisional Application No. 60/784,128, filed Mar. 20, 2006, which is incorporated herein by reference.

### GOVERNMENT SUPPORT

This invention was supported in part by a National Institutes of Health U.S. Department of Health and Human Services grant under Contract No. RO1CA98850. The Government may have certain rights in the invention.

### BACKGROUND OF THE INVENTION

1. Field of the Invention

This invention relates to the optimization of synthesis, resolution and purification of single isomers of substituted pyrimidine compounds and pharmaceutically acceptable salts, solvates and pro-drugs thereof. These single isomers have been found useful as anti-angiogenic agents for treating cancer and certain other diseases.

2. Description of the Prior Art

Racemic compounds are commonly used as therapeutic agents. However, the activity of the corresponding single isomers have been known to have diverse biological activity. Geometric isomers (E- and Z-isomers) and epimers are diastereomers as well as stereoisomers, having different spatial arrangements of atoms; consequently they are different compounds. As a result of their different configurations, their interactions with protein domains will be different. For example, the antipsychotic activity of doxepin Z-isomer was found to be significantly greater than the corresponding E-isomer. Since thalidomide has a chiral carbon atom, it exists as two enantiomers. Tests with mice suggested that only one enantiomer was teratogenic creating malformations in embryos while the other isomer possessed the therapeutic activity. Thus the preparation of single isomers of biologically active agents can be doubly beneficial in improving therapeutic responses while limiting adverse actions. In the present invention, methods for the preparation, resolution and isolation of E- and Z-isomers of 2,4-diamino-5-substituted vinylfuro[2,3-d]pyrimidine are revealed and the relevant biological activities of the isolated isomers are demonstrated.

Novel compounds were designed previously based on the known crystal structures of the VEGF receptor kinase and other factors, synthesized and assessed for biological activity (Gangjee, A et al., Bioorg. Med. Chem., 13:5475-91, 2005; U.S. Pat. No. 6,962,920). Design of these compounds was based on modifying a furopyrimidine scaffold expected to exhibit antifolate activity. To this scaffold, various functional groups were attached with the intent that the compounds would also bind to and inhibit the kinase domain of the VEGF receptor-2. Relevant testing established that certain of the compounds inhibited dihydrofolate reductase as well as having the ability to inhibit the VEGF and PDGF receptor kinases and inhibit angiogenesis in the chorioallantoic membrane assay. In designing these compounds a vinyl group was used to bridge the furopyrimidine scaffold to various attached groups in order that the functional groups align appropriately in a linear fashion into the kinase domain to maximize binding. The presence of the vinyl group in the molecule creates stereoisomerism, producing mixtures of compounds expected to vary in their chemical properties and biological activities as noted with other stereoisomeric mixtures.

Here we report on novel methods of synthesis and physical separation methods that allow the production of the individual stereoisomers. Their properties and activities will be revealed.

### SUMMARY OF THE INVENTION

The present invention provides methods of preparation of the individual isomer, E-2,4-substituted-5-vinylfuro[2,3-d]pyrimidine and respective pharmaceutically acceptable salts, solvates and prodrugs thereof, having the general formula (1):

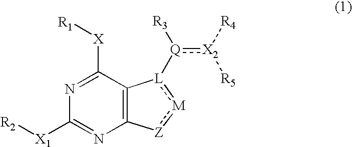

These were synthesized from 2,4-substituted-5-(chloromethyl)furo[2,3-d]pyrimidine and commercially available 2-substituted ketones using various reaction conditions and other reagents. Optimization of different reaction conditions, catalysis mediated chemistry, scale-up, isolation methods using chromatography and crystallization methods to separate the E- and Z-isomers and the biological activity will be discussed.

The formula (1) where X and X₁ are an alkyl group, an alkenyl group, a heteroaroyl group, a heteroalkyl group, a heteroalkenyl group or a heteroatom like N, S or O;
and R₁, and R₂ are a hydrogen, alkyl, alkenyl; aryl, aroyl, heteroalkyl, heteroalkenyl, heteroallyl group which can optionally be substituted with a mono-, di- and tri-substituted and unsubstituted aryl group like phenyl, 1-naphthyl, 2-naphthyl, and a straight, branched or cyclic lower alkyl group from 1 to about 6 carbons;
and R₃, R₄, or R₅ are the same or different and are selected from a group consisting of hydrogen, alkyl, alkenyl; aryl, aroyl, heteroalkyl, heteroalkenyl, heteroaroyl, heteroallyl group which can optionally be substituted with a mono-, di- and tri- substituted and unsubstituted aryl group like phenyl, 1-naphthyl, 2-naphthyl, and a straight, branched or cyclic lower alkyl group from 1 to about 6 carbons;
Z is selected from the group consisting of C, CH, CH₂, N, NH, S, O. L is selected from the group consisting of C, CH, CH₂, N, NH, S, O.CH═CH, CH═N and N═CH; but when Z is C, CH, CH═CH or CH₂ then L is N, NH, S or O;
M consists of CH;
the chemical bond between L and M is selected from the group consisting of a single bond and a double bond, and M is carbon when the bond is a double bond and M is CH when the bond is a single bond;

Q is selected from a group consisting of carbon, CH and CH₂;
the chemical bond between Q and X₂ is a double bond when R₃ is H or alkyl group; R₄ and R₅ are the same or different and are selected from group consisting of hydrogen, alkyl group, alkenyl group, heteroalkyl group, heteroaroyl group or heteroalkenyl group can optionally be substituted with a mono-, di- and tri-substituted and unsubstituted aryl group like phenyl, 1-naphthyl, 2-naphthyl, alkyl.aryl, aroyl, heteroalkyl, heteroalkeyl, heteroallyl and a straight, branched or cyclic lower alkyl group from 1 to about 6 carbons;
the chemical bond between M and Z is selected from the group consisting of a single bond and a double bond, and M is carbon when the bond is a double bond, and M is CH when the bond is a single bond. But when the bond between L and M is a double bond the bond between M and Z is a single bond.

It should be clear for one skilled in the art that if selective conditions are employed during the synthesis of the compounds it would be possible to achieve predominant synthesis of the desired isomer over the other.

In one aspect of the present invention, the pyrimidine compounds can function as receptor tyrosine kinase inhibitors, and resists the development of new blood vessels in tumors. Modeling studies suggest that the individual isomers will bind to the kinase domain of the VEGF receptor-2, but with different interactions and conformations between compound and receptor protein. Specifically, these compounds are usually tested as mixtures of steroyl isomers rather than as single isomers. The racemic mixtures have been found to inhibit receptor tyrosine kinases, including vascular endothelial growth factor (VEGF) and platelet derived growth factor (PDGF) receptor tyrosine kinases as well as inhibit dihydrofolate reductase. Thus, the compounds of the present invention are dual acting in that they can inhibit angiogenesis by inhibiting tyrosine kinases directly involved in angiogenesis, (such as by inhibiting the VEGF receptor tyrosine kinase), and inhibit the folate pathway required for cell growth. Our studies establish both quantitative and qualitative differences between the racemic mixture and the individual isomer in their activities. These compounds have an anti-angiogenic and an anti-tumor effect. In summary, the study encompasses the following steps:
Step 1. Synthesize compounds as a mixture of stereoisomers whose biological activities are assessed.
Step 2. Re-design the synthetic steps to achieve single isomers of the desired compound to be tested for biological activities.
Step 3. Resolve the isomers by physical or chromatographic methods and/or HPLC.

Comparison of the biological activity of individual geometric isomers showed significant differences in their activity from one another and the racemic mixture. For example, a pure E-isomer showed greater potency and a more persistent action than did the E/Z mixture of isomers in inhibiting capillary outgrowth in the aortic ring assay. Such results indicate that it may be beneficial for certain indications to use pure isomers rather than mixtures.

### DESCRIPTION OF THE PREFERRED EMBODIMENTS

The present invention provides methods of preparation of the individual isomer, E-2,4-substituted-5-vinylfuro[2,3-d]pyrimidine and respective pharmaceutically acceptable salts, solvates and prodrugs thereof, having the general formula (1):

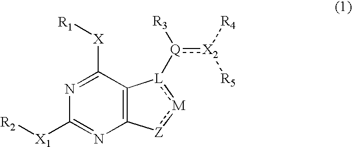

These were synthesized from 2,4-substituted-5-(chloromethyl)furo[2,3-d]pyrimidine and commercially available 2-substituted ketones using various reaction conditions and other reagents. Optimization of different reaction conditions, catalysis mediated chemistry, scale-up, isolation methods using chromatography and crystallization methods to separate the E- and Z-isomers and the biological activity will be discussed.

The formula (1) where X and X₁ are an alkyl group, an alkenyl group, a heteroaroyl group, a heteroalkyl group, a heteroalkenyl group or a heteroatom like N, S or O;
and R₁, and R₂ are a hydrogen, alkyl, alkenyl; aryl, aroyl, heteroalkyl, heteroalkenyl, heteroallyl group which can optionally be substituted with a mono-, di- and tri-substituted and unsubstituted aryl group like phenyl, 1-naphthyl, 2-naphthyl, and a straight, branched or cyclic lower alkyl group from 1 to about 6 carbons;
and R₃, R₄, or R₅ are the same or different and are selected from a group consisting of hydrogen, alkyl, alkenyl; aryl, aroyl, heteroalkyl, heteroalkenyl, heteroaroyl, heteroallyl group which can optionally be substituted with a mono-, di- and tri-substituted and unsubstituted aryl group like phenyl, 1-naphthyl, 2-naphthyl, and a straight, branched or cyclic lower alkyl group from 1 to about 6 carbons;
Z is selected from the group consisting of C, CH, CH₂, N, NH, S, O. L is selected from the group consisting of C, CH, CH₂, N, NH, S, O.CH═CH, CH═N and N═CH; but when Z is C, CH, CH═CH or CH₂ then L is N, NH, S or O;
M consists of CH;
the chemical bond between L and M is selected from the group consisting of a single bond and a double bond, and M is carbon when the bond is a double bond and M is CH when the bond is a single bond;
Q is selected from a group consisting of carbon, CH and CH₂;
the chemical bond between Q and X₂ is a double bond when R₃ is H or alkyl group; R₄ and R₅ are the same or different and are selected from group consisting of hydrogen, alkyl group, alkenyl group, heteroalkyl group, heteroaroyl group or heteroalkenyl group can optionally be substituted with a mono-, di- and tri-substituted and unsubstituted aryl group like phenyl, 1-naphthyl, 2-naphthyl, alkyl.aryl, aroyl, heteroalkyl, heteroalkeyl, heteroallyl and a straight, branched or cyclic lower alkyl group from 1 to about 6 carbons;
the chemical bond between M and Z is selected from the group consisting of a single bond and a double bond, and M is carbon when the bond is a double bond, and M is CH when the bond is a single bond. But when the bond between L and M is a double bond the bond between M and Z is a single bond.

It should be clear for one skilled in the art that if selective conditions are employed during the synthesis of the compounds it would be possible to achieve predominant synthesis of the desired isomer over the other.

In an embodiment of the present invention, the pyrimidine compounds can function as receptor tyrosine kinase inhibitors, and resists the development of new blood vessels in tumors (see Table 1 in Gangjee, A. et al., Bioorg. Med. Chem., 13:5475-91, 2005 and U.S. Pat. No. 6,962,920). Modeling studies suggest that the individual isomers will bind to the kinase domain of the VEGF receptor-2 but with different interactions and conformations between compound and receptor protein. Specifically, these compounds are usually tested as mixtures of steroyl isomers rather than as single isomers. The racemic mixtures have been found to inhibit receptor tyrosine kinases, including vascular endothelial growth factor (VEGF) and platelet derived growth factor (PDGF) receptor tyrosine kinases as well as inhibit dihydrofolate reductase. Thus, the compounds of the present invention are dual acting in that they can inhibit angiogenesis by inhibiting tyrosine kinases directly involved in angiogenesis, (such as by inhibiting the VEGF receptor tyrosine kinase), and inhibit the folate pathway required for cell growth. Our studies establish both quantitative and qualitative differences between the racemic mixture and the individual isomer in their activities. These compounds have an anti-angiogenic and an anti-tumor effect. In summary, the study encompasses the following steps:
Step 1. Synthesize compounds as a mixture of stereoisomers whose biological activities are assessed.
Step 2. Re-design the synthetic steps to achieve single isomers of the desired compound to be tested for biological activities.
Step 3. Resolve the isomers by physical or chromatographic methods and/or HPLC.

Comparison of the biological activity of individual geometric isomers showed significant differences in their activity from one another and the racemic mixture. For example, a pure E-isomer showed greater potency and a more persistent action than did the E/Z mixture of isomers in inhibiting capillary outgrowth in the aortic ring assay. Such results indicate that it may be beneficial for certain indications to use pure isomers rather than mixtures.

Whereas particular embodiments of this invention have been described above for purposes of illustration, it will be evident to those skilled in the art that numerous variations of the details of the present invention may be made without departing from the invention as defined in the appended claims.

1. A stereoselective method for preparing isolated E- and Z-isomers of 2,4-substituted-5-vinylfuro[2,3-d]pyrimidine and pharmaceutically acceptable salts, solvates and prodrugs thereof, comprising:
a. synthesizing said isolated E- and Z-isomers using at least one 2,4-substituted-5-(chloromethyl)furo[2,3-d]pyrimidine and at least one 2-substituted ketone using reaction conditions and reagents; and
b. separating said isolated E- and Z-isomers using at least one method selected from the group consisting of physical separation, chromatography and HPLC, wherein said isolated E- and Z-isomers each have the following composition: 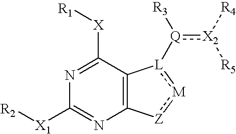
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkenyl, a heteroaroyl and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

2. The method according to claim 1 employing said isolated E- and Z-isomers, wherein said R₁ and said R₂ include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

3. The method according to claim 1 employing said isolated E- and Z-isomers, wherein said R₃, said R₄ and said R₅ include the same or different substituents selected from the group consisting essentially of mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

4. The method according to claim 1, for employing said isolated E- and Z-isomers, wherein said isolated E-isomers and Z-isomers inhibit at least one tyrosine kinase during angiogenesis.

5. The method according to claim 1 for employing said isolated E- and Z-isomers, wherein said isolated E-isomers and Z-isomers inhibit a folate pathway required for cell growth.

6. The method according to claim 1, wherein said isolated E-isomers and Z-isomers are anti-angiogenic agents.

7. The method according to claim for employing said isolated E- and Z-isomers, wherein said isolated E-isomers and Z-isomers are anti-cancer agents.

8. A composition comprising E-isomers and Z-isomers of 2,4-substituted-5-vinylfuro[2,3-d]pyrimidine and pharmaceutically acceptable salts, solvates, and prodrugs thereof, said composition of E-isomers and Z-isomers comprising: 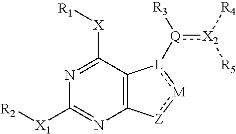
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkeyl, and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

9. The composition of claim 8, wherein said R₁ and said R₂ include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

10. The composition of claim 8, wherein said R₃, said R₄ and said R₅ include the same or different substituents selected from the group consisting essential of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

11. The composition of claim 8, wherein said composition inhibits two growth factors, said growth factors selected from the group consisting of VEGF and PDGF, and wherein the receptor for said VEGF is involved in the initial phases of angiogenesis and the receptor for said PDGF is involved in the stabilization of new capillaries.

12. The composition of claim 8, wherein a folate pathway is required for cell proliferation and VEGF and PDGF are required for inducing angiogenesis.

13. A method of therapeutic treatment of a disease, comprising:
employing isolated E-isomers and Z-isomers of 2,4-substituted-5-vinylfuro[2,3-d]pyrimidine and pharmaceutically acceptable salts, solvates, and prodrugs thereof comprising: 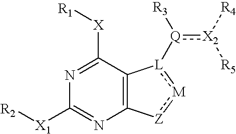
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkeyl, and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

14. The method of claim 13 employing said isolated E- and Z-isomers, wherein said R₁ and said R₂ include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

15. The method of claim 13 employing said isolated E- and Z-isomers, wherein said R₃, said R₄ and said R₅ include the same or different substituents selected from the group consisting essentially of mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

16. The method according to claim 13 for employing said isolated E- and Z-isomers, wherein said isolated E-isomers and Z-isomers inhibit at least one tyrosine kinase during angiogenesis.

17. The method according to claim 13 for employing said isolated E- and Z-isomers, wherein said isolated E-isomers and Z-isomers inhibit a folate pathway required for cell growth.

18. The method according to claim 13 for employing said isolated E- and Z-isomers, wherein either or both of said isolated E-isomers or said isolated Z-isomers are anti-angiogenic agents.

19. The method according to claim 13 for employing said isolated E- and Z-isomers, wherein said isolated E-isomers and Z-isomers are anti-cancer agents.

20. A method to reduce aberrant angiogenesis in diseases selected from the group consisting of rheumatoid arthritis, wet form of macular degeneration and cancer, comprising using either or both isolated E-isomer or isolated Z-isomer of the composition as claimed in claim 8.
